SEQUENCE LISTING (23 sequences)

SEQ ID NO: 1 - protein, 177 aa, homo sapiens
MEICRGLRSHLITLLLFLFHSETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 2 - protein, 153 aa, ARTIFICIAL
  Variant of human IL-1 receptor antagonist
MRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 3 - DNA, 473 bp, ARTIFICIAL
  Variant of human IL-1 receptor antagonist
catatgcgtccgagcggccgtaaaagcagcaaaatgcaggcgtttcgtatttgggatgtgaaccagaaaaccttttatctgcgtaacaaccagctggtggcgggctatctgcagggcccgaacgtgaacctggaagaaaaaattgatgtggtgccgattgaaccgcatgcgctgtttctgggcattcatggcggcaaaatgtgcctgagctgcgtgaaaagcggcgatgaaacccgtctgcagctggaagcggtgaacattaccgatctgagcgaaaaccgtaaacaggataaacgttttgcgtttattcgtagcgatagcggcccgaccaccagctttgaaagcgcggcgtgcccgggctggtttctgtgcaccgcgatggaagcggatcagccggtgagcctgaccaacatgccggatgaaggcgtgatggtgaccaaattttattttcaggaagatgaataataggatcc

SEQ ID NO: 4 - protein, 153 aa, ARTIFICIAL; identical to SEQ ID NO: 2
  Variant of human IL-1 receptor antagonist

SEQ ID NO: 5 - DNA, 465 bp, ARTIFICIAL
  Variant of human IL-1 receptor antagonist
atgagaccatctggtagaaaatcttctaaaatgcaagcttttagaatttgggatgttaatcaaaaaactttttatttgagaaataatcaattggttgctggttatttgcaaggtccaaatgttaatttggaagaaaaaattgatgttgttccaattgaaccacatgctttgtttttgggtattcatggtggtaaaatgtgtttgtcttgtgttaaatctggtgatgaaactagattgcaattggaagctgttaatattactgatttgtctgaaaatagaagacaagataaaagatttgcttttattagatctgattctggtccaactacttcttttgaatctgctgcttgtccaggttggtttttgtgtactgctatggaagctgatcaaccagtttctttgactaatatgccagatgaaggtgttatggttactaaattttattttcaagaagatgaataatag

SEQ ID NO: 6 - protein, 153 aa, ARTIFICIAL
  Variant of human IL-1 receptor antagonist
MRPSGRKSSKMQAFRIWDVNQKTFYLRNNQLVAGYLQGPNVNLEEKIDVVPIEPHALFLGIHGGKMCLSCVKSGDETRLQLEAVNITDLSENRRQDKRFAFIRSDSGPTTSFESAACPGWFLCTAMEADQPVSLTNMPDEGVMVTKFYFQEDE

SEQ ID NO: 7 - DNA, 465 bp, ARTIFICIAL
  Variant of human IL-1 receptor antagonist
atgagaccatctggtagaaaatcttctaaaatgcaagcttttagaatttgggatgttaatcaaaaaactttttatttgagaaataatcaattggttgctggttatttgcaaggtccaaatgttaatttggaagaaaaaattgatgttgttccaattgaaccacatgctttgtttttgggtattcatggtggtaaaatgtgtttgtcttgtgttaaatctggtgatgaaactagattgcaattggaagctgttaatattactgatttgtctgaaaatagaaaacaagatagaagatttgcttttattagatctgattctggtccaactacttcttttgaatctgctgcttgtccaggttggtttttgtgtactgctatggaagctgatcaaccagtttctttgactaatatgccagatgaaggtgttatggttactaaattttattttcaagaagatgaataatag

SEQ ID NO: 8 - protein, 153 aa, ARTIFICIAL
  Variant of human IL-1 receptor antagonist
MRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDRRFAFIRSDSGPTTSFESAACPGWFLCTAMEADQPVSLTNMPDEGVMVTKFYFQEDE

SEQ ID NO: 9 - DNA, 465 bp, ARTIFICIAL
  Variant of human IL-1 receptor antagonist
atgcgtccgagcggtcgtcgtagcagccgtatgcaggcatttcgtatttgggatgtgaatcagcgcaccttttatctgcgtaataatcagctggttgccggttatctgcagggtccgaatgttaatctggaagaacgcattgatgttgttccgattgaaccgcatgcactgtttctgggtattcatggtggtcgtatgtgtctgagctgtgttcgtagcggtgatgaaacccgtctgcagctggaagcagttaatattaccgatctgagcgaaaatcgtcgtcaggatcgtcgttttgcatttattcgtagcgatagcggtccgaccacctcttttgaaagcgcagcatgtccgggttggtttctgtgtaccgcaatggaagcagatcagccggttagcctgaccaatatgccggatgaaggtgttatggtgacccgcttttattttcaggaagatgaataatag

SEQ ID NO: 10 - protein, 153 aa, ARTIFICIAL
  Variant of human IL-1 receptor antagonist
MRPSGRRSSRMQAFRIWDVNQRTFYLRNNQLVAGYLQGPNVNLEERIDVVPIEPHALFLGIHGGRMCLSCVRSGDETRLQLEAVNITDLSENRRQDRRFAFIRSDSGPTTSFESAACPGWFLCTAMEADQPVSLTNMPDEGVMVTRFYFQEDE

SEQ ID NO: 11 - DNA, 465 bp, ARTIFICIAL
  Variant of human IL-1 receptor antagonist
atgcgtccgagcggtcgtcgtagcagccgtatgcaggcatttcgtatttgggatgtgaatcagcgcaccttttatctgcgtaataatcagctggttgccggttatctgcagggtccgaatgttaatctggaagaacgcattgatgttgttccgattgaaccgcatgcactgtttctgggtattcatggtggtcgtatgtgtctgagctgtgttcgtagcggtgatgaaacccgtctgcagctggaagcagttaacattaccgatctgagcgaaaatcgtaaacaggatcgtcgttttgcatttattcgtagcgatagcggtccgaccacctcttttgaaagcgcagcatgtccgggttggtttctgtgtaccgcaatggaagcagatcagccggttagcctgaccaatatgccggatgaaggtgttatggtgacccgcttttattttcaggaagatgaataatag

SEQ ID NO: 12 - protein, 153 aa, ARTIFICIAL
  Variant of human IL-1 receptor antagonist
MRPSGRRSSRMQAFRIWDVNQRTFYLRNNQLVAGYLQGPNVNLEERIDVVPIEPHALFLGIHGGRMCLSCVRSGDETRLQLEAVNITDLSENRKQDRRFAFIRSDSGPTTSFESAACPGWFLCTAMEADQPVSLTNMPDEGVMVTRFYFQEDE

SEQ ID NO: 13 - DNA, 465 bp, ARTIFICIAL
  Variant of human IL-1 receptor antagonist
atgcgtccgagcggtcgtcgtagcagccgtatgcaggcatttcgtatttgggatgtgaatcagcgcaccttttatctgcgtaataatcagctggttgccggttatctgcagggtccgaatgttaatctggaagaacgcattgatgttgttccgattgaaccgcatgcactgtttctgggtattcatggtggtcgtatgtgtctgagctgtgttaaaagcggtgatgaaacccgtctgcagctggaagcagttaatattaccgatctgagcgaaaatcgtcgtcaggatcgtcgttttgcatttattcgtagcgatagcggtccgaccacctcttttgaaagcgcagcatgtccgggttggtttctgtgtaccgcaatggaagcagatcagccggttagcctgaccaatatgccggatgaaggtgttatggtgacccgcttttattttcaggaagatgaataatag

SEQ ID NO: 14 - protein, 153 aa, ARTIFICIAL
  Variant of human IL-1 receptor antagonist
MRPSGRRSSRMQAFRIWDVNQRTFYLRNNQLVAGYLQGPNVNLEERIDVVPIEPHALFLGIHGGRMCLSCVKSGDETRLQLEAVNITDLSENRRQDRRFAFIRSDSGPTTSFESAACPGWFLCTAMEADQPVSLTNMPDEGVMVTRFYFQEDE

SEQ ID NO: 15 - DNA, 465 bp, ARTIFICIAL
  Variant of human IL-1 receptor antagonist
atgcgtccgagcggtcgtcgtagcagccgtatgcaggcatttcgtatttgggatgtgaatcagcgcaccttttatctgcgtaataatcagctggttgccggttatctgcagggtccgaatgttaatctggaagaacgcattgatgttgttccgattgaaccgcatgcactgtttctgggtattcatggtggtaaaatgtgtctgagctgtgttcgtagcggtgatgaaacccgtctgcagctggaagcagttaatattaccgatctgagcgaaaatcgtcgtcaggatcgtcgttttgcatttattcgtagcgatagcggtccgaccacctcttttgaaagcgcagcatgtccgggttggtttctgtgtaccgcaatggaagcagatcagccggttagcctgaccaatatgccggatgaaggtgttatggtgacccgcttttattttcaggaagatgaataatag

SEQ ID NO: 16 - protein, 153 aa, ARTIFICIAL
  Variant of human IL-1 receptor antagonist
MRPSGRRSSRMQAFRIWDVNQRTFYLRNNQLVAGYLQGPNVNLEERIDVVPIEPHALFLGIHGGKMCLSCVRSGDETRLQLEAVNITDLSENRRQDRRFAFIRSDSGPTTSFESAACPGWFLCTAMEADQPVSLTNMPDEGVMVTRFYFQEDE

SEQ ID NO: 17 - DNA, 465 bp, ARTIFICIAL
  Variant of human IL-1 receptor antagonist
atgcgtccgagcggtcgtcgtagcagccgtatgcaggcatttcgtatttgggatgtgaatcagcgcaccttttatctgcgtaataatcagctggttgccggttatctgcagggtccgaatgttaatctggaagaaaaaattgatgttgttccgattgaaccgcatgcactgtttctgggtattcatggtggtcgtatgtgtctgagctgtgttcgtagcggtgatgaaacccgtctgcagctggaagcagttaatattaccgatctgagcgaaaatcgtcgtcaggatcgtcgttttgcatttattcgtagcgatagcggtccgaccacctcttttgaaagcgcagcatgtccgggttggtttctgtgtaccgcaatggaagcagatcagccggttagcctgaccaatatgccggatgaaggtgttatggtgacccgcttttattttcaggaagatgaataatag

SEQ ID NO: 18 - protein, 153 aa, ARTIFICIAL
  Variant of human IL-1 receptor antagonist
MRPSGRRSSRMQAFRIWDVNQRTFYLRNNQLVAGYLQGPNVNLEEKIDVVPIEPHALFLGIHGGRMCLSCVRSGDETRLQLEAVNITDLSENRRQDRRFAFIRSDSGPTTSFESAACPGWFLCTAMEADQPVSLTNMPDEGVMVTRFYFQEDE

SEQ ID NO: 19 - protein, 25 aa, artificial
  Possible deletion from human IL-1 receptor antagonist
MEICRGLRSHLITLLLFLFHSETIC

SEQ ID NO: 20 - protein, 24 aa, artificial
  Possible deletion from human IL-1 receptor antagonist
MEICRGLRSHLITLLLFLFHSETI

SEQ ID NO: 21 - protein, 21 aa, artificial
  Possible deletion from human IL-1 receptor antagonist
MEICRGLRSHLITLLLFLFHS

SEQ ID NO: 22 - protein, 24 aa, artificial
  Possible addition to human IL-1 receptor antagonist
MALADLYEEGGGGGGEGEDNADSK

SEQ ID NO: 23 - protein, 34 aa, artificial
  Possible deletion from human IL-1 receptor antagonist
MEICRGLRSHLITLLLFLFHSETICRPSGRKSSK

(19) United States
(12) Patent Application Publication
(10) Pub. No.: US 2014/0011728 A1
(43) Pub. Date: Jan. 9, 2014

(54) LONG-ACTING IL-1 RECEPTOR ANTAGONISTS

(71) Applicants: Kristian Sass Drum, Koebenhavn N (DK); Thomas Hoeg-Jensen, Klampenborg (DK); Inga Sig Nielsen Noerby, Birkeroed (DK)

(72) Inventors: Kristian Sass Drum, Koebenhavn N (DK); Thomas Hoeg-Jensen, Klampenborg (DK); Inga Sig Nielsen Noerby, Birkeroed (DK)

(73) Assignee: Novo Nordisk A/S, Bagsvaerd (DK)

(21) Appl. No.: 13/982,340

(22) PCT Filed: Feb. 10, 2012
(86) PCT No.: PCT/EP2012/052317

§ 371 Date: Sep. 20, 2013

Related U.S. Application Data
(60) Provisional application No. 61/443,451, filed on Feb. 16, 2011.

(30) Foreign Application Priority Data
Feb. 15, 2011 (EP) 11154445.8

(51) Int. Cl. C07K 14/47

(52) U.S. Cl. CPC C07K 14/47
USPC 514/1.1; 530/409; 536/23.5; 435/320.1; 435/69.1; 435/252.33

(57) ABSTRACT

The present invention relates to IL-1 receptor antagonist compounds, compositions and use thereof as well as methods for preparation thereof.

### TECHNICAL FIELD

The present invention relates to therapeutic peptides, in particular protracted IL-1 receptor antagonists (IL-1Ra) as well as methods of their preparation, compositions and use thereof in medicine.

### BACKGROUND

IL-1 is produced by the body in response to inflammatory stimuli and mediates inflammatory responses. Inappropriately regulated signalling through the IL-1 receptor is known to promote severe conditions including, but not restricted to, rheumatoid arthritis and diabetes.

Anakinra (tradename Kineret), a recombinant non-glycosylated analogue of human IL-1Ra, is marketed for treatment of rheumatoid arthritis. Despite its benefits anakinra has important limitations. Firstly, anakinra is administered by injection, and patients experience pain, inflammation, and erythema at the injection site causing a proportion of new patients to discontinue therapy. Secondly, anakinra has a relatively short terminal half-life (4-6 hours) in the plasma, therefore one injection (100 mg) is typically required per day.

Elevated levels of IL-1 have been shown to damage and destroy insulin-producing cells. Islet IL-1β mRNA levels are up-regulated in type 2 diabetic patients, and elevated circulating glucose levels appear to contribute to the up-regulation. A recent study showed that antagonism of IL-1 with anakinra has a possible therapeutic potential in the treatment of type 2 diabetes; the treatment improved glycemia and beta-cell secretory function and reduced markers of systemic inflammation. Adipocyte functional studies have supported that IL-1 is able to impair insulin signalling.

Accordingly, it is desirable to provide an IL-1Ra with prolonged plasma half-life. Prolonged plasma half-life may result in clinical benefits, such as better coverage through 24 hours, and may allow less frequent dosing and thereby also reduce injection site reactions.

### SUMMARY

In some embodiments the invention relates to an IL-1 receptor antagonist (IL-1Ra) compound comprising an acylation group.

In some embodiments the invention relates to a composition comprising the IL-1Ra compound as defined herein and one or more excipients.

In some embodiments the invention relates to an IL-1Ra compound as defined herein for use in medicine.

In some embodiments the invention relates to a nucleotide sequence comprising a sequence selected from the group consisting of construct no. 2, construct no. 3, construct no. 4, construct no. 5, construct no. 6, construct no. 7 and construct no. 8. In some embodiments the invention relates to a vector comprising the nucleotide sequence as defined herein. In some embodiments the invention relates to a host cell comprising the nucleotide sequence as defined herein or the vector as defined herein. In some embodiments the invention relates to an amino acid sequence encoded by the nucleotide sequence as defined herein. In some embodiments the invention relates to a method for the preparation of an IL-1Ra compound, said method comprising the step of recombinant expression of a nucleotide sequence, such as a nucleotide sequence as defined herein, at a temperature below 25° C., such as at 18° C.

### BRIEF DESCRIPTION OF DRAWINGS

FIG. 1 shows SDS-PAGE analysis of recombinant expression of nucleotide-optimised IL-1Ra and variants thereof; U: un-induced, S: soluble protein fraction, P: insoluble protein fraction. FIG. 1A shows expression of construct no. 1 at 37° C. in three independent clones. FIG. 1B shows expression of construct no. 4-8 at 37° C. FIG. 1C shows expression of construct no. 4-7 at 37° C. vs. 18° C., wherein construct no. 4 and 5 were fermented at 37° C. for 3 hours and construct no. 6 and 7 were fermented at 18° C., over night. FIG. 1D shows expression of construct no. 2 and 3 in 800 mL shaking flask cultures induced by 0.4 mM IPTG and cultivated at 18° C., over night, I: induced, M: marker (14, 20, 30, 45, 66, 97 kDa).

### DESCRIPTION

The present invention relates to long-acting interleukin-1 receptor antagonist (IL-1Ra) compounds. In some embodiments the IL-1Ra compound comprises an acylation group.

In some embodiments the IL-1Ra compound of the present invention has an improved plasma half-life as determined after i.v. administration to rats or minipigs according to Assay (I) or Assay (II) described herein.

Surprisingly, the present inventors found that despite the presence of 9 Lys residues in anakinra, only a few Lys residues, such as one or two Lys residues, were targeted with the acylation chemistry used herein, see e.g. Examples 1 and 2. This enables feasible production of monocomponent Lys-acylated anakinra with fewer side products.

In some embodiments the nucleotide-optimised constructs, such as construct no. 1-8, of the present invention provides higher yield of soluble IL-1Ra compound.

In some embodiments expression of the nucleotide-optimised constructs, such as construct no. 1-8, at low temperature, such as less than 25° C., less than 20° C. or 18° C., provides higher yield of soluble IL-1Ra compound.

### IL-1Ra

In some embodiments the IL-1Ra compound comprises human IL-1Ra or an analogue thereof. The terms “human IL-1Ra”, “hIL-1Ra”, or “hIL-1Ra Isoform 1” are used interchangeably herein and refers to MEICRGLRSH LITLLLFLFH SETICRPSGR KSSKMQAFRI WDVNQKTFYL RNNQLVAGYL QGPNVNLEEK IDVVPIEPHA LFLGIHGGKM CLSCVKSGDE TRLQLEAVNI TDLSENRKQD KRFAFIRSDS GPTTSFESAA CPGWFLCTAM EADQPVSLTN MPDEGVMVTK FYFQEDE or refers to UNIPROT accession no. P18510-1. In some embodiments the N-terminal sequence MEICRGLRSH LITLLLFLFH SETIC of hIL-1Ra is deleted. In some embodiments the N-terminal sequence MEICRGLRSH LITLLLFLFH SETI of hIL-1Ra is deleted. In some embodiments hIL-1Ra comprises an N-terminal Met. In some embodiments hIL-1Ra comprises Met in position 25 (position relative to hIL-1Ra Isoform 1). Unless otherwise stated, references to positions in the IL-1Ra compound herein are relative to hIL-1Ra Isoform 1.

In some embodiments the N-terminal sequence MEICRGLRSH LITLLLFLFH S of hIL-1Ra is deleted and said hIL-1Ra comprises the N-terminal sequence MALADLYEEG GGGGGEGEDN ADSK (hIL-1Ra Isoform 2 UNIPROT accession no. P18510-2).

In some embodiments the N-terminal sequence MEICRGLRSH LITLLLFLFH S of hIL-1Ra is deleted and said hIL-1Ra comprises the N-terminal sequence MAL (hIL-1Ra Isoform 3 UNIPROT accession no. P18510-3).

In some embodiments the N-terminal sequence MEICRGLRSH LITLLLFLFH SETICRPSGR KSSK of hIL-1Ra is deleted (hIL-1Ra Isoform 4 UNIPROT accession no. P18510-4).

In some embodiments the IL-1Ra compound comprises anakinra or an analogue thereof. As used herein “anakinra” refers to [Met25]hIL-1Ra(25-177) or MRPSGRKSSK MQAFRIWDVN QKTFYLRNNQ LVAGYLQGPN VNLEEKIDVV PIEPHALFLG IHGGKMCLSC VKSGDETRLQ LEAVNITDLS ENRKQDKRFA FIRSDSGPTT SFESAACPGW FLCTAMEADQ PVSLTNMPDE GVMVTKFYFQ EDE, wherein Cys70 and Cys117 may be connected via a disulfide bond.

In some embodiments the IL-1Ra compound comprises one or more glycosylations.

The term “analogue” as used herein referring to a peptide means a modified peptide wherein one or more amino acid residues of the peptide have been substituted by other amino acid residues and/or wherein one or more amino acid residues of the peptide have been deleted from the peptide and/or one or more amino acid residues of the peptide have been added to the peptide. Such substitution of amino acid residues may take place in any position of the peptide, e.g. in position 31, 34, 118 and/or 121 (position relative to hIL-1Ra Isoform 1). Such addition or deletion of amino acid residues may take place at the N-terminal of the peptide and/or at the C-terminal of the peptide. A simple system is used to describe analogues, for example [Met25]hIL-1Ra(25-177) designates a hIL-1Ra Isoform 1 analogue wherein the amino acid sequence “MEICRGLRSHLITLLLFLFHSETI” in positions 1-24 of hIL-1Ra Isoform 1 has been deleted and the naturally occurring Cys in postion 25 has been substituted with Met.

In some embodiments the IL-1Ra compound is acylated in the N-terminal position and/or in Lys residues.

None of the 9 Lys-residues in human IL-1Ra have close contact to the human IL-1R1 receptor element, thus all of these could be potential targets for acylation without expected loss of affinity to the receptor.

In some embodiments the IL-1Ra compound comprises one or more Lys-residues are substituted into Arg. In some embodiments the IL-1Ra compound comprises the substitutions K31R, K34R, K118R and/or K121R (positions relative to hIL-1Ra Isoform 1).

### Acylation Group

The IL-1Ra compound of the invention comprises an acylation group.

In some embodiments an acyl group of a compound according to the present invention comprises a fatty acid or fatty diacid.

In some embodiments an acyl group of a compound according to the present invention comprises a fatty acid or fatty diacid and optionally a linker.

In some embodiments an acyl group of a compound according to the present invention comprises a fatty acid or fatty diacid and a linker.

In some embodiments a fatty acid or a fatty diacid of the acyl group according to the present invention comprises from 14, 16, 18, 20 or 22 amino acids.

In some embodiments a fatty acid or a fatty diacid of the acyl group according to the present invention comprises from 16, 18 or 20 amino acids.

In some embodiments a fatty acid or a fatty diacid of the acyl group according to the present invention comprises from 16, 18 or 22 amino acids.

In some embodiments the acylation group is a fatty acid substituent. A “fatty acid substituent” is herein understood as a side chain consisting of a fatty acid or a fatty diacid attached to the parent IL-1Ra, optionally via a linker, in an amino acid position such as a Lysine or a N-terminal amino acids.

In some embodiments, the “fatty acid substituent” attached to the parent IL-Ra has the general formula:

Acy-Lₙ-* (Formula I),

wherein n is 0 or an integer in the range from 1 to 10; Acy is a fatty acid or a fatty diacid comprising from about 14 to about 22 carbon atoms; L is an amino acid residue or a alkylene glycol moiety, wherein (*) designates the attachment site to IL-1Ra.

In some embodiments a fatty acid or a fatty diacid of the fatty acid substituent according to the present invention comprises from 14, 16, 18, 20 or 22 amino acids.

In some embodiments a fatty acid or a fatty diacid of the fatty acid substituent according to the present invention comprises from 16, 18 or 20 amino acids.

In some embodiments a fatty acid or a fatty diacid of the fatty acid substituent according to the present invention comprises from 16, 18 or 22 amino acids.

In some embodiments the acylation group comprises a carboxylic acid derivative selected from the group consisting of hexadecandioyl, octadecandioyl, eicosandioyl, and docosandioyl.

In some embodiments the compound of the present invention comprises fatty acid substituent according to formula (I), wherein Acy is selected from the group consisting of hexadecandioyl, octadecandioyl, eicosandioyl, and docosandioyl.

In some embodiments the acylation group comprises gamma-L-glutamate.

In some embodiments of the present invention AA1 of formula (I) is a linker.

In some embodiments of the present invention AA1 of formula (I) is a linker selected from the group consisting of D-γGlu, L-γGlu from which a hydrogen atom and/or a hydroxyl group has been removed.

In some embodiments of the present invention a linker according to the present invention is selected from the group consisting of D-γGlu, L-γGlu from which a hydrogen atom and/or a hydroxyl group has been removed.

In some embodiments the acylation group comprises one or more consecutive [2-(2-amino-ethoxy)-ethoxy]-acetyl, such as [2-(2-amino-ethoxy)-ethoxy]-acetyl-[2-(2-amino-ethoxy)-ethoxy]-acetyl.

In some embodiments of the present invention the fatty acid substituent according to the present invention is hexadecandioyl-gamma-L-glutamate.

In some embodiments the acylation group is hexadecandioyl-gamma-L-glutamate.

In some embodiments of the present invention the fatty acid substituent according to the present invention is octadecandioyl-gamma-L-glutamate-[2-(2-amino-ethoxy)-ethoxy]-acetyl-[2-(2-amino-ethoxy)-ethoxy]-acetyl.

In some embodiments the acylation group is octadecandioyl-gamma-L-glutamate-[2-(2-amino-ethoxy)-ethoxy]-acetyl-[2-(2-amino-ethoxy)-ethoxy]-acetyl.

In some embodiments of the present invention the fatty acid substituent according to the present invention is octadecandioyl-gamma-L-glutamate-[2-(2-amino-ethoxy)-ethoxy]-acetyl-[2-(2-amino-ethoxy)-ethoxy]-acetyl.

In some embodiments the acylation group is eicosandioyl-gamma-L-glutamate-[2-(2-amino-ethoxy)-ethoxy]-acetyl-[2-(2-amino-ethoxy)-ethoxy]-acetyl.

In some embodiments of the present invention the fatty acid substituent according to the present invention is eicosandioyl-gamma-L-glutamate-[2-(2-amino-ethoxy)-ethoxy]-acetyl-[2-(2-amino-ethoxy)-ethoxy]-acetyl.

In some embodiments the acylation group is docosandioyl-gamma-L-glutamate-[2-(2-amino-ethoxy)-ethoxy]-acetyl-[2-(2-amino-ethoxy)-ethoxy]-acetyl.

In some embodiments of the present invention the fatty acid substituent according to the present invention is docosandioyl-gamma-L-glutamate-[2-(2-amino-ethoxy)-ethoxy]-acetyl-[2-(2-amino-ethoxy)-ethoxy]-acetyl.

In some embodiments the acylation group is attached to IL-1Ra via the N-terminal amino group or a Lys amino acid residue of IL-1Ra. In some embodiments the acylation group is attached to IL-1Ra via the epsilon amino group of a Lys amino acid residue of the IL-1Ra compound.

In some embodiments the acylation group is attached to IL-1Ra via position 31, 34, 118 and/or 121 (positions relative to hIL-1Ra Isoform 1). In some embodiments the acylation group is attached to IL-1Ra via position 118 and/or 121 (positions relative to hIL-1Ra Isoform 1).

In some embodiments the IL-1Ra compound comprises a monoacylation with hexadecandioyl-gamma-L-glutamate in position 118 or 121 (positions relative to hIL-1Ra Isoform 1). As used herein the term “monoacylation” in the context of an IL-1Ra compound refers to a single acylation on said IL-1Ra compound.

In some embodiments the IL-1Ra compound comprises a monoacylation with octadecandioyl-gamma-L-glutamate-[2-(2-amino-ethoxy)-ethoxy]-acetyl-[2-(2-amino-ethoxy)-ethoxy]-acetyl in position 31, 34, 118 or 121 (position relative to hIL-1Ra Isoform 1).

In some embodiments the IL-1Ra compound comprises a monoacylation with eicosandioyl-gamma-L-glutamate-[2-(2-amino-ethoxy)-ethoxy]-acetyl-[2-(2-amino-ethoxy)-ethoxy]-acetyl in position 118 or 121 (position relative to hIL-1Ra Isoform 1).

The acylation group may be prepared as described in WO2005/012347, WO2009/115469 or WO2006/084842.

In some embodiments the IL-1Ra compound comprises hIL-1Ra Isoform 1 with N-hexadecandioyl-gamma-L-glutamate in position K118 or K121 (compound A).

In some embodiments the IL-1Ra compound is hIL-1Ra Isoform 1 with N-octadecandioyl-gamma-L-glutamate in position K118 (compound B).

In some embodiments the IL-1Ra compound is hIL-1Ra Isoform 1 with N-octadecandioyl-gamma-L-glutamate-[2-(2-amino-ethoxy)-ethoxy]-acetyl-[2-(2-amino-ethoxy)-ethoxy]-acetyl] in position K118 (compound B).

In some embodiments the IL-1Ra compound is hIL-1Ra Isoform 1 with N-octadecandioyl-gamma-L-glutamate-[2-(2-amino-ethoxy)-ethoxy]-acetyl-[2-(2-amino-ethoxy)-ethoxy]-acetyl] in position K31 or K34 (compound C).

In some embodiments the IL-1Ra compound is hIL-1Ra Isoform 1 with N-eicosandioyl-gamma-L-glutamate-[2-(2-amino-ethoxy)-ethoxy]-acetyl-[2-(2-amino-ethoxy)-ethoxy]-acetyl] in position K118 or K121 (compound D).

In some embodiments the IL-1Ra compound is selected from the group consisting of
N-epsilon118-[N-hexadecandioyl-gamma-L-glutamate][Met25]hIL-1Ra(25-177) (compound A1),
N-epsilon121-[N-hexadecandioyl-gamma-L-glutamate][Met25]hIL-1Ra(25-177) (compound A2), or a mixture thereof.

In some embodiments the IL-1Ra compound is selected from the group consisting of
N-epsilon118-[N-octadecandioyl-gamma-L-glutamyl-[2-(2-amino-ethoxy)-ethoxy]-acetyl-[2-(2-amino-ethoxy)-ethoxy]-acetyl][Met25]hIL-1Ra(25-177) (compound B),
N-epsilon31-[N-octadecandioyl-gamma-L-glutamyl-[2-(2-amino-ethoxy)-ethoxy]-acetyl-[2-(2-amino-ethoxy)-ethoxy]-acetyl][Met25]hIL-1Ra(25-177) (compound C1),
N-epsilon34-[N-octadecandioyl-gamma-L-glutamyl-[2-(2-amino-ethoxy)-ethoxy]-acetyl-[2-(2-amino-ethoxy)-ethoxy]-acetyl][Met25]hIL-1Ra(25-177) (compound C2), or a mixture thereof.

In some embodiments the IL-1Ra compound is selected from the group consisting of N-epsilon118-[N-eicosandioyl-gamma-L-glutamyl-[2-(2-amino-ethoxy)-ethoxy]-acetyl-[2-(2-amino-ethoxy)-ethoxy]-acetyl][Met25]hIL-1Ra(25-177) (compound D1), N-epsilon121-[N-eicosandioyl-gamma-L-glutamyl-[2-(2-amino-ethoxy)-ethoxy]-acetyl-[2-(2-amino-ethoxy)-ethoxy]-acetyl][Met25]hIL-1Ra(25-177) (compound D2), or a mixture thereof.

### Functional Features

In some embodiments the IL-1Ra compound has an in vivo plasma half-life of at least 1.5, such as at least 2 or at least 3, times the half-life of anakinra. In some embodiments the IL-1Ra compound has an in vivo plasma half-life of at least 5, such as at least 10, times the half-life of anakinra. Unless otherwise is stated “in vivo plasma half-life” as used herein means in vivo plasma half-life determined according to Assay (I) or Assay (II) described herein.

In some embodiments the IL-1Ra compound protects against IL-1β-activity as determined by Assay (III) described herein.

### Methods of Preparation

In some embodiments the IL-1Ra compound may be prepared by recombinant expression as described in Example 5. Recombinant expression induced at lower temperatures, such as 18° C., provided a higher yield of recombinant IL-1Ra. Recombinant expression induced at lower temperatures, such as 18° C., provided soluble recombinant IL-1Ra. Accordingly, in some embodiments the invention relates to a method for the preparation of an IL-1Ra compound, said method comprising the step of recombinant expression of a nucleotide sequence, such as a nucleotide sequence as defined in claim 11, at a temperature below 25° C., such as at 18° C.

In some embodiments the invention relates to a nucleotide sequence comprising a sequence selected from the group consisting of construct no. 2, construct no. 3, construct no. 4, construct no. 5, construct no. 6, construct no. 7 and construct no. 8.

In some embodiments the invention relates to a vector comprising the nucleotide sequence of the invention.

In some embodiments the invention relates to a host cell comprising the nucleotide sequence or the vector of the invention.

In some embodiments the invention relates to an amino acid sequence encoded by the nucleotide sequence of the invention.

In some embodiments the invention relates to a method for the preparation of an IL-1Ra compound comprising the steps of
a) nucleotide sequence optimization,
b) preparation of expression plasmid,
c) transformation into a host cell,
d) cultivation of expression host cells harbouring expression plasmid and
e) subsequent capture and purification of recombinant IL-1Ra compound.

### Pharmaceutical Compositions

In some embodiments the invention relates to a composition comprising the IL-1Ra compound and one or more pharmaceutically acceptable excipients. In some embodiments the composition comprises one or more IL-1Ra compounds, such as two IL-1Ra compounds.

### Pharmaceutical Indications

The compound of the invention may be used in medicine. In some embodiments the invention relates to the IL-1Ra compound for treatment of diabetes, rheumatoid arthritis, systemic juvenile idiopathic arthritis (SJIA), cardiovascular disease, gout, inflammatory bowel disease (IBD), lupus erythematosus (SLE), Alzheimer's disease, multiple sclerosis, deficiency of the IL1 receptor antagonist (DIRA), or Muckle-Wells syndrome.

In some embodiments the invention relates to a method for the treatment of diabetes, rheumatoid arthritis, systemic juvenile idiopathic arthritis (SJIA), cardiovascular disease, gout, inflammatory bowel disease (IBD), lupus erythematosus (SLE), Alzheimer's disease, multiple sclerosis, deficiency of the IL1 receptor antagonist (DIRA), Muckle-Wells syndrome or other conditions, wherein control of IL-1 signalling has a clinical benefit by administration of the compound according to the invention, by administration of the compound of the invention.

### Definitions

The term “human IL-1Ra analogue” as used herein means a modified human IL-1Ra wherein one or more amino acid residues of the IL-1Ra have been substituted by other amino acid residues and/or wherein one or more amino acid residues have been deleted from the IL-1Ra and/or wherein one or more amino acid residues have been added and/or inserted to the IL-1Ra.

In some embodiments an IL-1Ra analogue comprises less than 10 amino acid modifications (substitutions, deletions, additions (including insertions) and any combination thereof) relative to IL-1Ra, alternatively less than 9, 8, 7, 6, 5, 4, 3, 2 or 1 modification relative to IL-1Ra.

Herein, the term “amino acid residue” is an amino acid from which, formally, a hydroxy group has been removed from a carboxy group and/or from which, formally, a hydrogen atom has been removed from an amino group.

The term “IL-1Ra derivative” as used herein means a chemically modified parent IL-1Ra or an analogue thereof, wherein the modification(s) are in the form of attachment of amides, carbohydrates, alkyl groups, acyl groups, esters, PEGylations, and the like.

“An IL-1Ra” according to the invention is herein to be understood as human IL-1Ra or an IL-1Ra from another species such as porcine or bovine insulin.

The term “IL-1Ra peptide” as used herein means a peptide which is either human IL-1Ra or an analog or a derivative thereof with insulin activity.

The term “parent IL-1Ra” as used herein is intended to mean an IL-1Ra before any modifications according to the invention have been applied thereto.

### Mode of Administration.

Herein, the term “acylated IL-1Ra” covers modification of IL-1Ra by attachment of one or more fatty acid substituents optionally via a linker to the IL-1Ra peptide.

In one aspect an insulin peptide used in a composition according to the invention is N-terminally modified and furthermore substituted with a fatty acid substituent in a position other than one of the N-terminals of the insulin, wherein the fatty acid substituent consists of a fatty acid or a difatty acid attached to the insulin optionally via a linker. The linker may be any suitable portion in between the fatty acid or the fatty diacid and the point of attachment to the insulin, which portion may also be referred to as a linker moiety, spacer, or the like.

The term “treatment” is meant to include both the prevention and minimization of the referenced disease, disorder, or condition (i.e., “treatment” refers to both prophylactic and therapeutic administration IL-1Ra derivative or composition comprising IL-1Ra derivative unless otherwise indicated or clearly contradicted by context.

The route of administration may be any route which effectively transports a compound of this invention to the desired or appropriate place in the body, such as parenterally, for example, subcutaneously, intramuscularly or intraveneously. Alternatively, a compound of this invention can be administered orally, pulmonary, rectally, transdermally, buccally, sublingually, or nasally.

For parenterally administration, a compound of this invention is formulated analogously with the formulation of known insulins. Furthermore, for parenterally administration, a compound of this invention is administered analogously with the administration of known insulins and the physicians are familiar with this procedure.

Herein, the term “fatty acid” covers a linear or branched, aliphatic carboxylic acids having at least two carbon atoms and being saturated or unsaturated. Non limiting examples of fatty acids are myristic acid, palmitic acid, and stearic acid.

Herein, the term “fatty diacid” covers a linear or branched, aliphatic dicarboxylic acids having at least two carbon atoms and being saturated or unsaturated. Non limiting examples of fatty diacids are hexanedioic acid, octanedioic acid, decanedioic acid, dodecanedioic acid, tetradecanedioic acid, hexadecanedioic acid, heptadecanedioic acid, octadecanedioic acid, and eicosanedioic acid.

The term “about” as used herein means in reasonable vicinity of the stated numerical value, such as plus or minus 10%.

### EXAMPLES

### Synthesis of Intermediates

The acylation group, e.g. N-hexadecandioyl-gamma-L-glutamyl succinimidyl ester, N-octadecandioyl-gamma-L-glutamyl-[2-(2-amino-ethoxy)-ethoxy]-acetyl-[2-(2-amino-ethoxy)-ethoxy]-acetyl succinimidyl ester, and N-eicosandioyl-gamma-L-glutamyl-[2-(2-amino-ethoxy)-ethoxy]-acetyl-[2-(2-amino-ethoxy)-ethoxy]-acetyl succinimidyl ester may be prepared as described in WO2005/012347, WO2009/115469 or WO02010/029159.

### Example 1

### Met25]hIL-1Ra(25-177) with N-hexadecandioyl-gamma-L-glutamate in Position K118 or K121 (Compound A)

Derivatisation of [Met25]hIL-1Ra(25-177) with N-hexadecandioyl-gamma-L-glutamyl succinimidyl ester (Chem. 1).

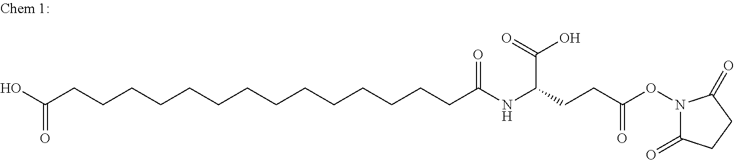 [SMILES: O=C(O)CCCCCCCCCCCCCCC(=O)N[C@@H](CCC(=O)ON1C(=O)CCC1=O)C(=O)O]

Kineret (660 μL, 5.8 nmol anakinra, commercially available from Amgen) was diluted with 5% NaHCO3, pH 8 (1340 μL), pH of mixture was 7. N-hexadecandioyl-gamma-L-glutamyl succinimidyl ester (4.8 mg, 5.8 nmol) was dissolved in DMF (300 μL) and added to the protein solution. LCMS analysis after 15 minutes showed formation of product as: 1963 Da [M+9H]9+, 1359 Da [M+13]13+, 1262Da [M+14H]14+. The acylated protein was purified on a Source 30Q anion exchange column (20 mM Tris pH 8.0, 50 to 200 mM NaCl over 20 column volumes) and stored at 4° C.

Surprisingly, the anion exchange chromatography purified product was [Met25]hIL-1Ra(25-177) monoacylated at K118 or K121 (compound A). Proteolytic Asp-N degradation followed by MALDI-TOF MS or LCMS analysis showed the product to be a mixture of
N-epsilon118-[N-hexadecandioyl-gamma-L-glutamate][Met25]hIL-1Ra(25-177) (compound A1), and
N-epsilon121-[N-hexadecandioyl-gamma-L-glutamate][Met25]hIL-1Ra(25-177) (compound A2).

### Example 2

### [Met25]hIL-1Ra(25-177) with N-octadecandioyl-gamma-L-glutamyl-[2-(2-amino-ethoxy)-ethoxy]-acetyl-[2-(2-amino-ethoxy)-ethoxy]-acetyl in Position K118 (Compound B), or in Position K31 or K34 (Compound C)

Derivatisation of [Met25]hIL-1Ra(25-177) with N-octadecandioyl-gamma-L-glutamyl-[2-(2-amino-ethoxy)-ethoxy]-acetyl-[2-(2-amino-ethoxy)-ethoxy]-acetyl succinimidyl ester (Chem. 2)

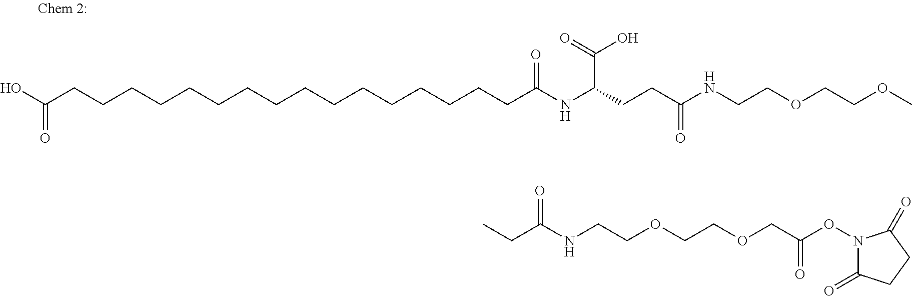

Kineret (660 μL, 5.8 nmol anakinra, commercially available from Amgen) was diluted with 5% NaHCO3, pH 8 (1340 μL), pH of mixture was 7. N-octadecandioyl-gamma-L-glutamyl-[2-(2-amino-ethoxy)-ethoxy]-acetyl-[2-(2-amino-ethoxy)-ethoxy]-acetyl succinimidyl ester (4.8 mg, 5.8 nmol) was dissolved in DMF (300 μL) and added to the protein solution. LCMS analysis after 15 minutes showed formation of product as: 1998 Da [M+9H]9+, 1799 Da [M+10H]10+, 1635 Da [M+11H]11+, 1499 Da [M+12H]12+, 1383 Da [M+13]13+. The acylated protein was purified on a Source 30Q anion exchange column (20 mM Tris pH 8.0, 50 to 200 mM NaCl over 20 column volumes) and stored at 4° C.

Surprisingly, separation by anion exchange chromatography resulted in a major acylation product (compound B), which was shown by Asp-N degradation and LCMS to be acylated in position K118 of [Met25]hIL-1Ra(25-177) (>80% of the product in this chromatographic pool), and a minor acylation product (compound C), which was monoacylated in position K31 or K34 of [Met25]hIL-1Ra(25-177). The products were a mixture of
N-epsilon118-[N-octadecandioyl-gamma-L-glutamyl-[2-(2-amino-ethoxy)-ethoxy]-acetyl-[2-(2-amino-ethoxy)-ethoxy]-acetyl][Met25]hIL-1Ra(25-177) (compound B),
N-epsilon31-[N-octadecandioyl-gamma-L-glutamyl-[2-(2-amino-ethoxy)-ethoxy]-acetyl-[2-(2-amino-ethoxy)-ethoxy]-acetyl] [Met25]hIL-1Ra(25-177) (compound C1), and
N-epsilon34-[N-octadecandioyl-gamma-L-glutamyl-[2-(2-amino-ethoxy)-ethoxy]-acetyl-[2-(2-amino-ethoxy)-ethoxy]-acetyl][Met25]hIL-1Ra(25-177) (compound C2).

### Example 3

### [Met25]hIL-1Ra(25-177) with N-eicosandioyl-gamma-L-glutamate-[2-(2-amino-ethoxy)-ethoxy]-acetyl-[2-(2-amino-ethoxy)-ethoxy]-acetyl in Position K118 or K121 (Compound D)

Derivatisation of [Met25]hIL-1Ra(25-177) with N-eicosandioyl-gamma-L-glutamate-[2-(2-amino-ethoxy)-ethoxy]-acetyl-[2-(2-amino-ethoxy)-ethoxy]-acetyl succinimidyl ester (Chem. 3).

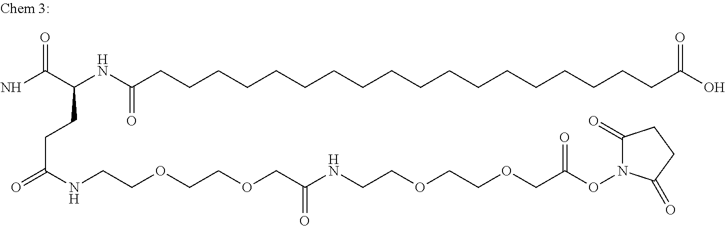 [SMILES: NC(=O)[C@H](CCC(=O)NCCOCCOCC(=O)NCCOCCOCC(=O)ON1C(=O)CCC1=O)NC(=O)CCCCCCCCCCCCCCCCCCC(=O)O]

Kineret (1.32 mL, 11.6 nmol anakinra, commercially available from Amgen) was diluted with 0.1 M NaHCO3, pH 8 (6.65 mL), pH of mixture was 7.4. N-eicosandioyl-gamma-L-glutamyl-[2-(2-amino-ethoxy)-ethoxy]-acetyl-[2-(2-amino-ethoxy)-ethoxy]-acetyl succinimidyl ester (10.0 mg, 11.6 nmol) was dissolved in DMF (1.5 mL) and added to the protein solution. LCMS analysis after 15 minutes showed formation of acylated product, [M+12 H]⁺=1501.2 Da, calculated 1501.0 Da, which was purified by methods as described for compounds A-C.

The purified product was monoacylated [Met25]hIL-1Ra(25-177) (compound D). Proteolytic Asp-N degradation followed by MALDI-TOF MS or LCMS analysis showed compound D to be a mixture of two predominant products,
N-epsilon118-[N-eicosandioyl-gamma-L-glutamate-[2-(2-amino-ethoxy)-ethoxy]-acetyl-[2-(2-amino-ethoxy)-ethoxy]-acetyl][Met25]hIL-1Ra(25-177) (compound D1) and
N-epsilon121-[N-eicosandioyl-gamma-L-glutamate-[2-(2-amino-ethoxy)-ethoxy]-acetyl-[2-(2-amino-ethoxy)-ethoxy]acetyl][Met25]hIL-1Ra(25-177) (compound D2).

### Biological Assays

### Assay (I): Plasma Half-Life Determined in Rats

The following assay is useful for evaluating the pharmacokinetics in rats. The rats were dosed by intravenous (IV) administration in a sparse sampling study design. Male Sprague-Dawley rats (Taconic) weighing approx. 250 g were dosed IV with 1-10 μg test substance, dissolved in Gibco DPBS w/o Ca and Mg (GIBCO cat. no. 14190), per animal. The animals had ad libitum access to food and water and were acclimatized for at least one week before entering the study. Blood (650 μl) was sampled from the vena sublingualis (tongue) without anaesthesia at e.g. 5 minutes, 15 minutes, 30 minutes, 1 hour, 2 hours, 4 hours, 8 hours and 24 hours post dosing. Each rat was sampled four times and after the last sampling, the rats were sacrificed. Blood was collected in tubes containing EDTA. Samples were kept on ice and centrifuged (5 min, 4° C., 8000 rpm). Plasma samples of 2×150 μL were transferred into micronic tubes for each time point and kept at −20° C. until analysis. The plasma samples were analysed by Assay (A) as described below.

### Assay (II): Plasma Half-Life Determined in Pigs

The following assay is useful for evaluating the pharmacokinetics in pigs. The pigs were dosed by intravenous (IV) administration. Male Göttingen mini-pigs (Ellegaard Göttingen Minipigs A/S, Denmark) weighing approx 15-30 kg were dosed IV via a venflon inserted in the ear vein. Blood was sampled from the jugular vein. Test substances were dissolved in Gibco DPBS w/o Ca and Mg (GIBCO catalog nummer 14190) and the dose was 0.2 mg per animal. Blood samples were taken at e.g. the following time points: pre-dose, 5 minutes, 15 minutes, 30 minutes, 45 minutes, 1 hour, 1.5 hours, 2 hours, 3 hours, 4 hours, 6 hours, 8 hours, 10 hours, 24 hours, 48 hours, 72 hours, 96 hours, 120 hours, 168 hours, 192 hours, 216 hours, 240 hours, 264 hours and 288 hours post dosing. The blood samples (0.8 mL) were collected into test tubes containing EDTA buffer for stabilization and kept on ice for max. 20 minutes before centrifugation. The centrifugation procedure to separate plasma may be: 4° C. and approx. 2500 g for 10 minutes. Plasma was collected and immediately transferred (2×200 μL) to Micronic tubes stored at −20° C. until assayed. The plasma samples were analysed by Assay (A) as described below.

### Assay (A): Quantitative Assay for Plasma Samples

Quantitative Assay for Plasma Samples: Measurement of IL-1Ra compounds in plasma was conducted using a commercial available ELISA kit (Quantikine kit from R&D Systems) for human interleukin 1 receptor antagonist (IL-1Ra). The ELISA was a sandwich immunoassay, based on one monoclonal and one polyclonal antibody. At all times the assay procedure from R&D Systems was followed except 1) the kit standard was used as controls, 2) standards of each IL-1Ra compound were prepared in specie plasma correspondent to the samples (rat or minipig) and 3) sample dilution were in specie plasma. The protocol in short was: The monoclonal antibody specific for IL-1Ra was pre-coated onto a microplate. Standards of each IL-1Ra compound were prepared in plasma, the kit standard and samples were pipetted into the wells and any IL-1Ra present was bound by the immobilized antibody. After washing away any unbound substances, an enzyme-linked polyclonal antibody specific for IL-1Ra was added to the wells. Following a wash to remove any unbound antibody-enzyme reagent, a substrate solution was added to the wells and colour developed in proportion to the amount of IL-1Ra bound in the initial step. The colour development was stopped and the intensity of the colour was measured.

Non-compartmental analysis (NCA): Plasma concentration-time profiles were analyzed by non-compartmental pharmacokinetics analysis (NCA) using WinNonlin (Pharsight Inc., Mountain View, Calif., USA). NCA was performed using the individual plasma concentration-time profiles from each animal.

### Assay (III): NO-Production

INS-1 cells are derived from a rat insulinoma and are widely used in studies of beta-cell. INS-1 cells express the IL-1 receptor and activation of this receptor by IL-1β leads to increased iNOS expression and subsequently to nitric oxide (NO) release. NO is very unstable and quickly breaks down to the more stable by-products nitrate and nitrite. Nitrite levels in the medium can easily be measured using the Griess method and is thus an indirect measure of the NO production. In the present experiment, INS-1 cells were seeded in 96 well plates and incubated at 37° C. The day after, 150 μg/ml IL-1β and increasing concentrations of IL-1 receptor antagonist compounds were added to the cells and the plates are incubated at 37° C. for 48 hours. After the incubation the medium was analysed for NO using the Griess method.

### Example 4

### Plasma Half-Life of IL-1Ra Compounds

Plasma half-life of anakinra and Compound A, B and C was determined using Assay (I) and Assay (II). Dosage to rats was 1 μg (anakinra), 5 μg (compounds B and C), or 10 μg (compound A). Dosage to minipigs was 200 μg anakinra, Compound A, B or C. The results are shown in Table 1.

TABLE 1
Plasma half-life of anakinra and acylated anakinra compounds

| | | | Rat PK | Pig PK |
|---|---|---|---|---|
| IL-1Ra | | Acylated | T½ | T½ |
| compound | Acylation group | residue^{a)} | (min) | (min) |
| Anakinra | — | — | 67¹⁾ | 80 |
| Compound A | Hexadecandioyl-gamma-L-glutamyl | K118 or | 113 | 270 |
| | | K121²⁾ | | |
| Compound B | Octadecandioyl-gamma-L-glutamyl-[2- | K118³⁾ | 216 | 840 |
| | (2-amino-ethoxy)-ethoxy]-acetyl-[2- | | | |
| | (2-amino-ethoxy)-ethoxy]-acetyl | | | |
| Compound C | Octadecandioyl-gamma-L-glutamyl-[2- | K31 or K34⁴⁾ | 222 | 474 |
| | (2-amino-ethoxy)-ethoxy]-acetyl-[2- | | | |
| | (2-amino-ethoxy)-ethoxy]-acetyl | | | |

^{a)}Position relative to hIL-1Ra Isoform 1
¹⁾Average of two independent experiments
²⁾Product contained ~50% of each isomer
³⁾More than 80% of acylation was on this residue
⁴⁾Acylation was on these residues only

The results showed that acylation improved the rat plasma half-life from 67 to as much as 222 minutes (compound C) and rat plasma half-life from 80 to as much as 840 minutes (compound B).

### Example 5

### NO-Production from IL-1Ra Compounds

NO-production of IL-1Ra compounds was determined according to Assay (III). Measurements were made in quadruplicate. The results are shown in Table 2.

TABLE 2
NO-production from IL-1Ra compounds

| IL-1Ra | NO release (μM) | | | |
|---|---|---|---|---|
| (ng/ml) | Anakinra | Compound A | Compound B | Compound C |
| 0 | 6.56 | 6.53 | 6.51 | 6.60 |
| 5 | 0.54 | 4.88 | 5.60 | 5.56 |
| 10 | −0.63 | 3.76 | 5.29 | 5.46 |
| 50 | −0.84 | −0.76 | 2.08 | 3.67 |
| 100 | −0.68 | −0.74 | 0.27 | 2.36 |

### Assay (IV): Acute Inhibitory Effect on Release of Inflammatory Markers in Mice

Six to seven weeks old C57bl/female mice (n=6 for all groups) were intraperitonally administered anakinra (10 or 100 mg/kg; injection volume 5 ml/kg) at time −½ hour, or Compound C (10, 30 or 100 mg/kg; injection volume 5 ml/kg) at time −1 hour, before treatment at time 0 hour with hIL-1 (1 ng/g, R&D Systems; subcutaneous injection volume 10 ml/kg). At time 2 hours all mice were anaesthetised with isoflurane and blood collected from the abdominal aorta. The plasma level of inflammatory markers IL-6 and MCP-1 was measured using xMAP multiplex assay (Luminex). The results are shown in Table 3.

TABLE 3
Acute inhibitory effect on release of inflammatory markers in mice

| | | Anakinra | | Compound C | | |
|---|---|---|---|---|---|---|
| | | 10 | 100 | 10 | 30 | 100 |
| | Vehicle | mg/kg | mg/kg | mg/kg | mg/kg | mg/kg |
| IL-6 (pg/ml) | 594 | 14 | 10 | 25 | 17 | 11 |
| (StdDev) | (317) | (8) | (4) | (11) | (7) | (7) |
| MCP-1 (pg/ml) | 1157 | 6 | 5 | 8 | 7 | 6 |
| (StdDev) | (1091) | (2) | (2) | (4) | (3) | (4) |

### Methods of Preparation

### Example 6

### Recombinant Expression of IL-1Ra or Variants Thereof in E. coli and Construction of Encoding DNA Sequences

In order to improve the recombinant IL-1Ra expression system the DNA encoding IL-1Ra and recombinant IL-1Ra variants were nucleotide-optimized. The nucleotide-optimized IL-1Ra and IL-1Ra-variants are shown in Table 3.

The IL-1Ra encoding DNA fragment was isolated NdeI+BamHI from the nucleotide-optimized IL-1Ra or IL-1Ra-variant encoding DNA fragments and subcloned in NdeI+BamHI restricted pET11a.

For expression the IL-1Ra_pET11a construct was transformed into BL21(DE3) and expression was induced by addition of 0.4 mM IPTG and continued at 37° C. for 3 hours or 18° C. over-night. The expected molecular weight was approx. 17 kDa. At 37° C., expression of soluble recombinant IL-1Ra-variants was scarse, however, a significantly larger soluble IL-1Ra-fraction was observed following induction at 18° C. over-night.

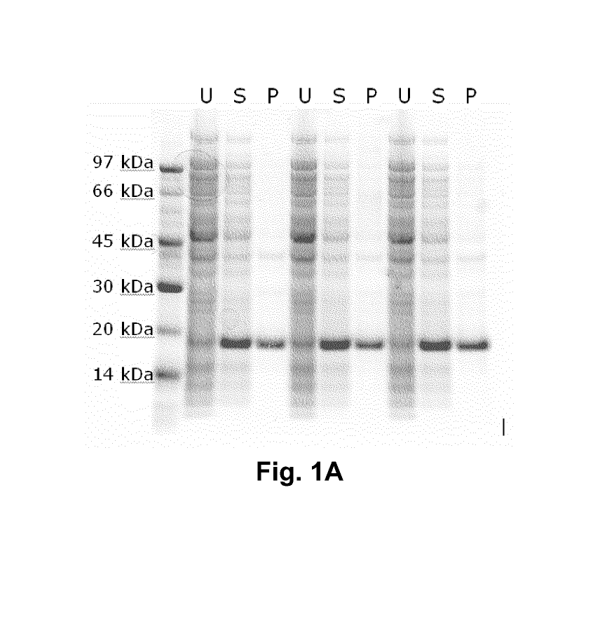

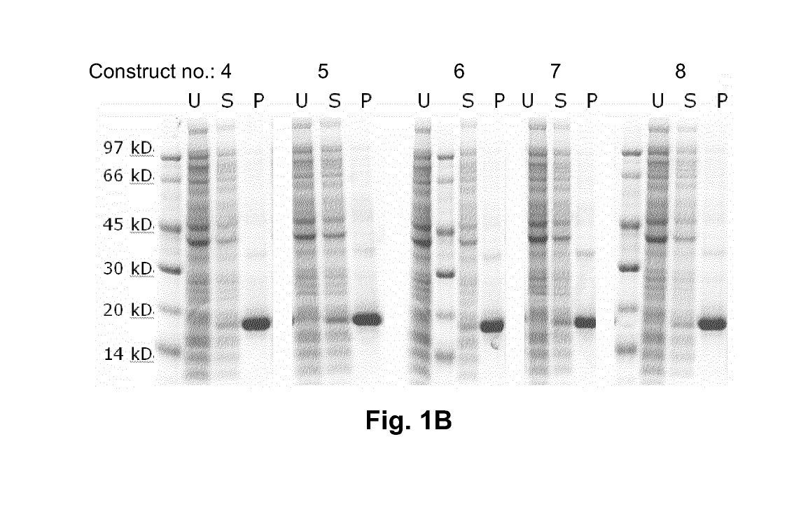

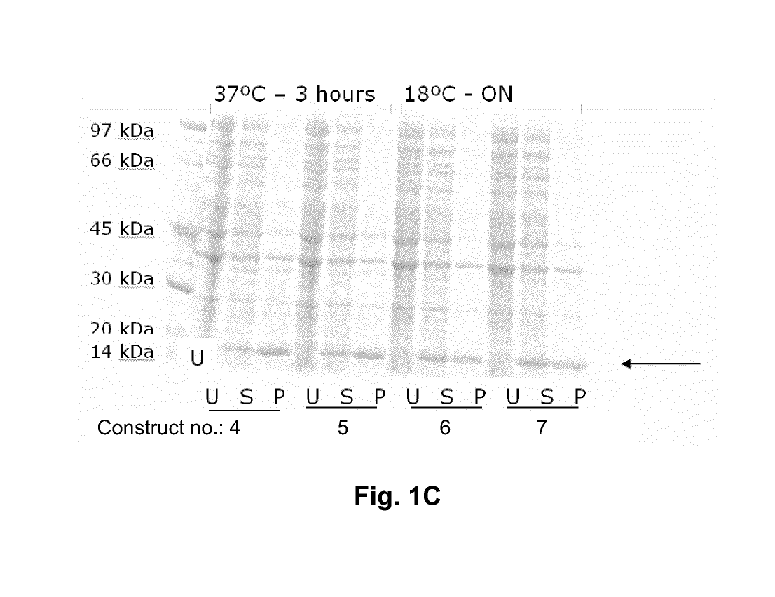

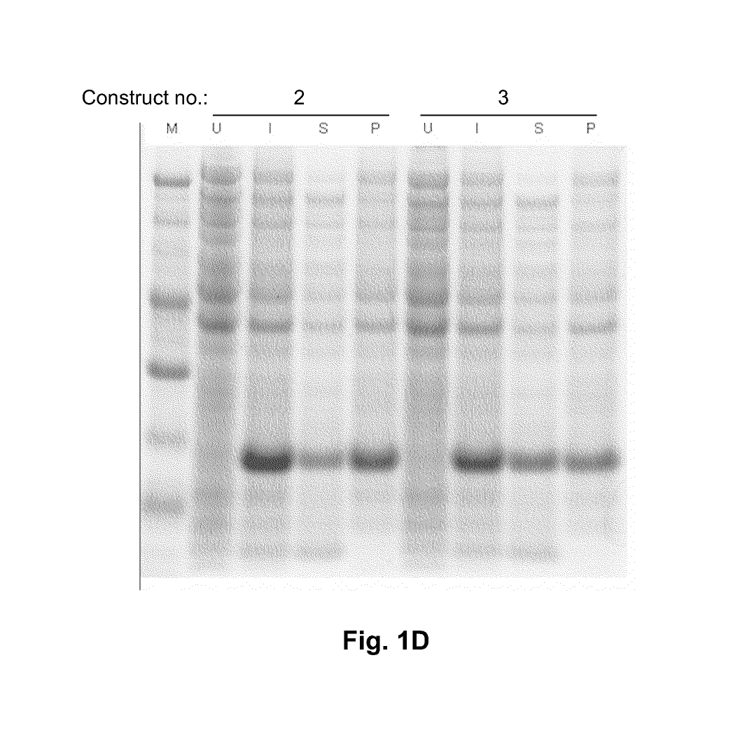

Expression was analysed by SDS-PAGE, the results are shown in FIG. 1. FIG. 1A shows expression of construct no. 1 at 37° C. in three independent clones. FIG. 1B shows expression of construct no. 4-8 at 37° C. FIG. 1C shows expression of construct no. 4-7 at 37° C. vs. 18° C., wherein construct no. 4 and 5 were fermented at 37° C. for 3 hours and construct no. 6 and 7 were fermented at 18° C., over night. FIG. 1D shows expression of construct no. 2 and 3 in 800 mL shaking flask cultures induced by 0.4 mM IPTG and cultivated at 18° C., over night, I; induced, M: marker (14, 20, 30, 45, 66, 97 kDa).

Yields in 5 mL batch cultures were 120-160 mg/liter of soluble recombinant IL-1Ra using construct no. 1.

It was found that the nucleotide-optimized encoding DNA sequence provided optimal recombinant IL1RA expression levels in E. coli. In particular, not only were expression levels much increased, the yield of soluble protein was also increased. Furthermore, it was found that for some constructs expression induced at lower temperatures, such as 18° C., provided a higher yield of recombinant IL-1Ra and maintained the recombinant IL-1Ra soluble.

TABLE 3
Overview of constructs of IL-1Ra (construct no. 1) and
IL-1Ra-variant (construct no. 2-8) amino acid sequences
and encoding DNA sequences. The length of the nucleotide
sequences of construct no. 1-8 is 473 nucleotides.

| Construct | | |
|---|---|---|
| no. | Name, amino acid sequence and encoding DNA sequence | |
| 1 | [Met25]hIL-1Ra(25-177) | |
| | | NdeI |
| | | ~~~~~~~ |
| | | M R P S G R K S S K M Q A F R I W |
| | 1 | CATA TGCGTCCGAG CGGCCGTAAA AGCAGCAAAA TGCAGGCGTT |
| | | TCGTATTTGG |
| | | D V N Q K T F Y L R N N Q L V A G Y L Q |
| | 61 | GATGTGAACC AGAAAACCTT TTATCTGCGT AACAACCAGC TGGTGGCGGG |
| | | CTATCTGCAG |
| | | G P N V N L E E K I D V V P I E P H A L |
| | 121 | GGCCCGAACG TGAACCTGGA AGAAAAAATT GATGTGGTGC CGATTGAACC |
| | | GCATGCGCTG |
| | | F L G I H G G K M C L S C V K S G D E T |
| | 181 | TTTCTGGGCA TTCATGGCGG CAAAATGTGC CTGAGCTGCG TGAAAAGCGG |
| | | CGATGAAACC |
| | | R L Q L E A V N I T D L S E N R K Q D K |
| | 241 | CGTCTGCAGC TGGAAGCGGT GAACATTACC GATCTGAGCG AAAACCGTAA |
| | | ACAGGATAAA |
| | | R F A F I R S D S G P T T S F E S A A C |
| | 301 | CGTTTTGCGT TTATTCGTAG CGATAGCGGC CCGACCACCA GCTTTGAAAG |
| | | CGCGGCGTGC |
| | | P G W F L C T A M E A D Q P V S L T N M |
| | 361 | CCGGGCTGGT TTCTGTGCAC CGCGATGGAA GCGGATCAGC CGGTGAGCCT |
| | | GACCAACATG |
| | | BamHI |
| | | ~~~~~~ |
| | | P D E G V M V T K F Y F Q E D E * * |
| | 421 | CCGGATGAAG GCGTGATGGT GACCAAATTT TATTTTCAGG AAGATGAATA |
| | | ATAGGATCC |
| 2 | [Met24, Arg118]hIL-1Ra(24-177) | |
| | | M R P S G R K S S K M Q A F R I W D V |
| | 1 | ATGAGAC CATCTGGTAG AAAATCTTCT AAAATGCAAG CTTTTAGAAT |
| | | TTGGGATGTT |
| | | N Q K T F Y L R N N Q L V A G Y L Q G P |
| | 61 | AATCAAAAAA CTTTTTATTT GAGAAATAAT CAATTGGTTG CTGGTTATTT |
| | | GCAAGGTCCA |
| | | N V N L E E K I D V V P I E P H A L F L |
| | 121 | AATGTTAATT TGGAAGAAAA AATTGATGTT GTTCCAATTG AACCACATGC |
| | | TTTGTTTTTG |
| | | G I H G G K M C L S C V K S G D E T R L |
| | 181 | GGTATTCATG GTGGTAAAAT GTGTTTGTCT TGTGTTAAAT CTGGTGATGA |
| | | AACTAGATTG |
| | | Q L E A V N I T D L S E N R R Q D K R F |
| | 241 | CAATTGGAAG CTGTTAATAT TACTGATTTG TCTGAAAATA GAAGACAAGA |
| | | TAAAAGATTT |
| | | A F I R S D S G P T T S F E S A A C P G |
| | 301 | GCTTTTATTA GATCTGATTC TGGTCCAACT ACTTCTTTTG AATCTGCTGC |
| | | TTGTCCAGGT |
| | | W F L C T A M E A D Q P V S L T N M P D |
| | 361 | TGGTTTTTGT GTACTGCTAT GGAAGCTGAT CAACCAGTTT CTTTGACTAA |
| | | TATGCCAGAT |
| | | E G V M V T K F Y F Q E D E * * |
| | 421 | GAAGGTGTTA TGGTTACTAA ATTTTATTTT CAAGAAGATG AATAATAG |
| 3 | [Met24, Arg121]hIL-1Ra(24-177) | |
| | | M R P S G R K S S K M Q A F R I W D V |
| | 1 | ATGAGAC CATCTGGTAG AAAATCTTCT AAAATGCAAG CTTTTAGAAT |
| | | TTGGGATGTT |
| | | N Q K T F Y L R N N Q L V A G Y L Q G P |
| | 61 | AATCAAAAAA CTTTTTATTT GAGAAATAAT CAATTGGTTG CTGGTTATTT |
| | | GCAAGGTCCA |
| | | N V N L E E K I D V V P I E P H A L F L |
| | 121 | AATGTTAATT TGGAAGAAAA AATTGATGTT GTTCCAATTG AACCACATGC |
| | | TTTGTTTTTG |
| | | G I H G G K M C L S C V K S G D E T R L |
| | 181 | GGTATTCATG GTGGTAAAAT GTGTTTGTCT TGTGTTAAAT CTGGTGATGA |
| | | AACTAGATTG |
| | | Q L E A V N I T D L S E N R K Q D R R F |
| | 241 | CAATTGGAAG CTGTTAATAT TACTGATTTG TCTGAAAATA GAAAACAAGA |
| | | TAGAAGATTT |
| | | A F I R S D S G P T T S F E S A A C P G |
| | 301 | GCTTTTATTA GATCTGATTC TGGTCCAACT ACTTCTTTTG AATCTGCTGC |
| | | TTGTCCAGGT |
| | | W F L C T A M E A D Q P V S L T N M P D |
| | 361 | TGGTTTTTGT GTACTGCTAT GGAAGCTGAT CAACCAGTTT CTTTGACTAA |
| | | TATGCCAGAT |
| | | E G V M V T K F Y F Q E D E * * |
| | 421 | GAAGGTGTTA TGGTTACTAA ATTTTATTTT CAAGAAGATG AATAATAG |
| 4 | [Met24, Arg31, Arg34, Arg46, Arg70, Arg89, | |
| | Arg96, Arg118, Arg121, Arg170]-hIL-1Ra(24-177) | |
| | | M R P S G R R S S R M Q A F R I W |
| | 1 | A TGCGTCCGAG CGGTCGTCGT AGCAGCCGTA TGCAGGCATT |
| | | TCGTATTTGG |
| | | D V N Q R T F Y L R N N Q L V A G Y L Q |
| | 61 | GATGTGAATC AGCGCACCTT TTATCTGCGT AATAATCAGC TGGTTGCCGG |
| | | TTATCTGCAG |
| | | G P N V N L E E R I D V V P I E P H A L |
| | 121 | GGTCCGAATG TTAATCTGGA AGAACGCATT GATGTTGTTC CGATTGAACC |
| | | GCATGCACTG |
| | | F L G I H G G R M C L S C V R S G D E T |
| | 181 | TTTCTGGGTA TTCATGGTGG TCGTATGTGT CTGAGCTGTG TTCGTAGCGG |
| | | TGATGAAACC |
| | | R L Q L E A V N I T D L S E N R R Q D R |
| | 241 | CGTCTGCAGC TGGAAGCAGT TAATATTACC GATCTGAGCG AAAATCGTCG |
| | | TCAGGATCGT |
| | | R F A F I R S D S G P T T S F E S A A C |
| | 301 | CGTTTTGCAT TTATTCGTAG CGATAGCGGT CCGACCACCT CTTTTGAAAG |
| | | CGCAGCATGT |
| | | P G W F L C T A M E A D Q P V S L T N M |
| | 361 | CCGGGTTGGT TTCTGTGTAC CGCAATGGAA GCAGATCAGC CGGTTAGCCT |
| | | GACCAATATG |
| | | P D E G V M V T R F Y F Q E D E * * |
| | 421 | CCGGATGAAG GTGTTATGGT GACCCGCTTT TATTTTCAGG AAGATGAATA ATAG |
| 5 | [Met24, Arg31, Arg34, Arg46, Arg70, Arg89, | |
| | Arg96, Arg121, Arg170]-hIL-1Ra(24-177) | |
| | | M R P S G R R S S R M Q A F R I W |
| | 1 | A TGCGTCCGAG CGGTCGTCGT AGCAGCCGTA TGCAGGCATT |
| | | TCGTATTTGG |
| | | D V N Q R T F Y L R N N Q L V A G Y L Q |
| | 61 | GATGTGAATC AGCGCACCTT TTATCTGCGT AATAATCAGC TGGTTGCCGG |
| | | TTATCTGCAG |
| | | G P N V N L E E R I D V V P I E P H A L |
| | 121 | GGTCCGAATG TTAATCTGGA AGAACGCATT GATGTTGTTC CGATTGAACC |
| | | GCATGCACTG |
| | | F L G I H G G R M C L S C V R S G D E T |
| | 181 | TTTCTGGGTA TTCATGGTGG TCGTATGTGT CTGAGCTGTG TTCGTAGCGG |
| | | TGATGAAACC |
| | | R L Q L E A V N I T D L S E N R K Q D R |
| | 241 | CGTCTGCAGC TGGAAGCAGT TAACATTACC GATCTGAGCG AAAATCGTAA |
| | | ACAGGATCGT |
| | | R F A F I R S D S G P T T S F E S A A C |
| | 301 | CGTTTTGCAT TTATTCGTAG CGATAGCGGT CCGACCACCT CTTTTGAAAG |
| | | CGCAGCATGT |
| | | P G W F L C T A M E A D Q P V S L T N M |
| | 361 | CCGGGTTGGT TTCTGTGTAC CGCAATGGAA GCAGATCAGC CGGTTAGCCT |
| | | GACCAATATG |
| | | P D E G V M V T R F Y F Q E D E * * |
| | 421 | CCGGATGAAG GTGTTATGGT GACCCGCTTT TATTTTCAGG AAGATGAATA ATAG |
| 6 | [Met24, Arg31, Arg34, Arg46, Arg70, Arg89, | |
| | Arg118, Arg121, Arg170]-hIL-1Ra(24-177) | |
| | | M R P S G R R S S R M Q A F R I W |
| | 1 | A TGCGTCCGAG CGGTCGTCGT AGCAGCCGTA TGCAGGCATT |
| | | TCGTATTTGG |
| | | D V N Q R T F Y L R N N Q L V A G Y L Q |
| | 61 | GATGTGAATC AGCGCACCTT TTATCTGCGT AATAATCAGC TGGTTGCCGG |
| | | TTATCTGCAG |
| | | G P N V N L E E R I D V V P I E P H A L |
| | 121 | GGTCCGAATG TTAATCTGGA AGAACGCATT GATGTTGTTC CGATTGAACC |
| | | GCATGCACTG |
| | | F L G I H G G R M C L S C V K S G D E T |
| | 181 | TTTCTGGGTA TTCATGGTGG TCGTATGTGT CTGAGCTGTG TTAAAAGCGG |
| | | TGATGAAACC |
| | | R L Q L E A V N I T D L S E N R R Q D R |
| | 241 | CGTCTGCAGC TGGAAGCAGT TAATATTACC GATCTGAGCG AAAATCGTCG |
| | | TCAGGATCGT |
| | | R F A F I R S D S G P T T S F E S A A C |
| | 301 | CGTTTTGCAT TTATTCGTAG CGATAGCGGT CCGACCACCT CTTTTGAAAG |
| | | CGCAGCATGT |
| | | P G W F L C T A M E A D Q P V S L T N M |
| | 361 | CCGGGTTGGT TTCTGTGTAC CGCAATGGAA GCAGATCAGC CGGTTAGCCT |
| | | GACCAATATG |
| | | P D E G V M V T R F Y F Q E D E * * |
| | 421 | CCGGATGAAG GTGTTATGGT GACCCGCTTT TATTTTCAGG AAGATGAATA ATAG |
| 7 | [Met24, Arg31, Arg34, Arg46, Arg70, Arg96, | |
| | Arg118, Arg121, Arg170]-hIL-1Ra(24-177) | |
| | | M R P S G R R S S R M Q A F R I W |
| | 1 | A TGCGTCCGAG CGGTCGTCGT AGCAGCCGTA TGCAGGCATT |
| | | TCGTATTTGG |
| | | D V N Q R T F Y L R N N Q L V A G Y L Q |
| | 61 | GATGTGAATC AGCGCACCTT TTATCTGCGT AATAATCAGC TGGTTGCCGG |
| | | TTATCTGCAG |
| | | G P N V N L E E R I D V V P I E P H A L |
| | 121 | GGTCCGAATG TTAATCTGGA AGAACGCATT GATGTTGTTC CGATTGAACC |
| | | GCATGCACTG |
| | | F L G I H G G K M C L S C V R S G D E T |
| | 181 | TTTCTGGGTA TTCATGGTGG TAAAATGTGT CTGAGCTGTG TTCGTAGCGG |
| | | TGATGAAACC |
| | | R L Q L E A V N I T D L S E N R R Q D R |
| | 241 | CGTCTGCAGC TGGAAGCAGT TAATATTACC GATCTGAGCG AAAATCGTCG |
| | | TCAGGATCGT |
| | | R F A F I R S D S G P T T S F E S A A C |
| | 301 | CGTTTTGCAT TTATTCGTAG CGATAGCGGT CCGACCACCT CTTTTGAAAG |
| | | CGCAGCATGT |
| | | P G W F L C T A M E A D Q P V S L T N M |
| | 361 | CCGGGTTGGT TTCTGTGTAC CGCAATGGAA GCAGATCAGC CGGTTAGCCT |
| | | GACCAATATG |
| | | P D E G V M V T R F Y F Q E D E * * |
| | 421 | CCGGATGAAG GTGTTATGGT GACCCGCTTT TATTTTCAGG AAGATGAATA ATAG |
| 8 | [Met24, Arg31, Arg34, Arg46, Arg89, Arg96, | |
| | Arg118, Arg121, Arg170]-hIL-1Ra(24-177) | |
| | | M R P S G R R S S R M Q A F R I W |
| | 1 | A TGCGTCCGAG CGGTCGTCGT AGCAGCCGTA TGCAGGCATT |
| | | TCGTATTTGG |
| | | D V N Q R T F Y L R N N Q L V A G Y L Q |
| | 61 | GATGTGAATC AGCGCACCTT TTATCTGCGT AATAATCAGC TGGTTGCCGG |
| | | TTATCTGCAG |
| | | G P N V N L E E K I D V V P I E P H A L |
| | 121 | GGTCCGAATG TTAATCTGGA AGAAAAAATT GATGTTGTTC CGATTGAACC |
| | | GCATGCACTG |
| | | F L G I H G G R M C L S C V R S G D E T |
| | 181 | TTTCTGGGTA TTCATGGTGG TCGTATGTGT CTGAGCTGTG TTCGTAGCGG |
| | | TGATGAAACC |
| | | R L Q L E A V N I T D L S E N R R Q D R |
| | 241 | CGTCTGCAGC TGGAAGCAGT TAATATTACC GATCTGAGCG AAAATCGTCG |
| | | TCAGGATCGT |
| | | R F A F I R S D S G P T T S F E S A A C |
| | 301 | CGTTTTGCAT TTATTCGTAG CGATAGCGGT CCGACCACCT CTTTTGAAAG |
| | | CGCAGCATGT |
| | | P G W F L C T A M E A D Q P V S L T N M |
| | 361 | CCGGGTTGGT TTCTGTGTAC CGCAATGGAA GCAGATCAGC CGGTTAGCCT |
| | | GACCAATATG |
| | | P D E G V M V T R F Y F Q E D E * * |
| | 421 | CCGGATGAAG GTGTTATGGT GACCCGCTTT TATTTTCAGG AAGATGAATA ATAG |

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

### The Following is a Non-Limiting List of Aspect Further Comprised within the Scope of the Invention

1. An IL-1 receptor antagonist (IL-1Ra) compound comprising an acylation group.
2. The IL-1Ra compound according to any one of the preceding aspects, wherein said acylation group is attached to IL-1Ra via position 31, 34, 118 and/or 121 (positions relative to hIL-1Ra Isoform 1).
3. The IL-1Ra compound according to any one of the preceding aspects, wherein said IL-1Ra compound comprises i) a monoacylation with N-hexadecandioyl-gamma-L-glutamyl in position 118 or 121 (positions relative to hIL-1Ra Isoform 1) or ii) a monoacylation with N-octadecandioyl-gamma-L-glutamyl-[2-(2-amino-ethoxy)-ethoxy]-acetyl-[2-(2-amino-ethoxy)-ethoxy]-acetyl at 31, 34, 118 or 121 (positions relative to hIL-1Ra Isoform 1), or iii) a monoacylation with N-eicosandioyl-gamma-L-glutamyl-[2-(2-amino-ethoxy)-ethoxy]-acetyl-[2-(2-amino-ethoxy)-ethoxy]-acetyl at 118 or 121 (positions relative to hIL-1Ra Isoform 1).
4. The IL-1Ra compound according to any one of the preceding aspects, wherein said acylation group is selected from the group consisting of N-hexadecandioyl-gamma-L-glutamyl, N-octadecandioyl-gamma-L-glutamyl-[2-(2-amino-ethoxy)-ethoxy]-acetyl-[2-(2-amino-ethoxy)-ethoxy]-acetyl, N-eicosandioyl-gamma-L-glutamyl-[2-(2-amino-ethoxy)-ethoxy]-acetyl-[2-(2-amino-ethoxy)-ethoxy]-acetyl, and N-docosandioyl-gamma-L-glutamyl-[2-(2-amino-ethoxy)-ethoxy]-acetyl-[2-(2-amino-ethoxy)-ethoxy]-acetyl.
5. The IL-1Ra compound according to any one of the preceding aspects, wherein said IL-1Ra is human IL-1Ra or an analogue thereof.
6. The IL-1Ra compound according to any one of the preceding aspects, wherein said acylation group is attached to IL-1Ra via the epsilon amino group of a Lys amino acid residue of said IL-1Ra.
7. The IL-1Ra compound according to any one of the preceding aspects, wherein said IL-1Ra comprises the substitutions K31R, K34R, K118R and/or K121R (positions relative to hIL-1Ra Isoform 1).
8. The IL-1Ra compound according to any one of the preceding aspects, wherein said IL-1Ra compound is selected from the group consisting of N-epsilon118-[N-hexadecandioyl-gamma-L-glutamate][Met25]hIL-1Ra(25-177) (compound A1), N-epsilon121-[N-hexadecandioyl-gamma-L-glutamate][Met25]hIL-1Ra(25-177) (compound A2), a mixture of compound Al and A2, N-epsilon118-[N-octadecandioyl-gamma-L-glutamyl-[2-(2-amino-ethoxy)-ethoxy]-acetyl-[2-(2-amino-ethoxy)-ethoxy]-acetyl][Met25]hIL-1Ra(25-177) (compound B), N-epsilon31-[N-octadecandioyl-gamma-L-glutamyl-[2-(2-amino-ethoxy)-ethoxy]-acetyl-[2-(2-amino-ethoxy)-ethoxy]-acetyl][Met25]hIL-1Ra(25-177) (compound C1), N-epsilon34-[N-octadecandioyl-gamma-L-glutamyl-[2-(2-amino-ethoxy)-ethoxy]-acetyl-[2-(2-amino-ethoxy)-ethoxy]-acetyl][Met25]hIL-1Ra(25-177) (compound C2), N-epsilon118-[N-eicosandioyl-gamma-L-glutamyl-[2-(2-amino-ethoxy)-ethoxy]-acetyl-[2-(2-amino-ethoxy)-ethoxy]-acetyl][Met25]hIL-1Ra(25-177) (compound D1), N-epsilon118-[N-eicosandioyl-gamma-L-glutamyl-[2-(2-amino-ethoxy)-ethoxy]-acetyl-[2-(2-amino-ethoxy)-ethoxy]-acetyl][Met25]hIL-1Ra(25-177) (compound D2), a mixture of compound C1 and C2, a mixture of compound B, C1 and C2, or a mixture of compound D1 and D2
9. A composition comprising the IL-1Ra compound as defined in any one of the preceding aspects and one or more excipients.
10. The IL-1Ra compound according to any one of the preceding aspects for use in medicine.
11. A nucleotide sequence comprising a sequence selected from the group consisting of construct no. 2, construct no. 3, construct no. 4, construct no. 5, construct no. 6, construct no. 7 and construct no. 8.
12. A vector comprising the nucleotide sequence as defined in aspect 11.
13. A host cell comprising the nucleotide sequence as defined in aspect 11 or the vector as defined in aspect 12.
14. An amino acid sequence encoded by the nucleotide sequence as defined in aspect 11.
15. A method for the preparation of an IL-1Ra compound, said method comprising the step of recombinant expression of a nucleotide sequence, such as a nucleotide sequence as defined in aspect 11, at a temperature below 25° C., such as at 18° C.

1. An IL-1 receptor antagonist (IL-1Ra) compound, wherein said IL-1Ra is human IL-1Ra or an analogue thereof and comprises an acylation group, wherein said acylation group comprises a fatty acid or fatty diacid.

2. An IL-1 receptor antagonist (IL-1Ra) compound according to claim 1, further comprising a linker.

3. An IL-1 receptor antagonist (IL-1Ra) compound comprising an acylation group wherein said acylation group comprises a lipophilic substituent of formula I.
Acy-Lₙ-* (Formula I),
wherein n is 0 or an integer in the range from 1 to 10; Acy is a fatty acid or a fatty diacid comprising from about 8 to about 24 carbon atoms; L is an amino acid residue or a alkylene glycol moiety and * designates the attachment site to IL-1Ra.

4. The IL-1Ra compound according to claim 1, wherein said acylation group is attached to IL-1Ra at an amino acid selected from the group consisting of position 31, 34, 118 and 121 relative to hIL-1Ra Isoform 1.

5. The IL-1Ra compound according to claim 1, wherein said IL-1Ra compound comprises i) a monoacylation with N-hexadecandioyl-gamma-L-glutamyl in position 118 or 121 (positions relative to hIL-1Ra Isoform 1) or ii) a monoacylation with N-octadecandioyl-gamma-L-glutamyl-[2-(2-amino-ethoxy)-ethoxy]-acetyl-[2-(2-amino-ethoxy)-ethoxy]-acetyl at 31, 34, 118 or 121 (positions relative to hIL-1Ra Isoform 1) or iii) a monoacylation with N-eicosandioyl-gamma-L-glutamyl-[2-(2-amino-ethoxy)-ethoxy]-acetyl-[2-(2-amino-ethoxy)-ethoxy]-acetyl at 118 or 121 (positions relative to hIL-1Ra Isoform.

6. The IL-1Ra compound according to claim 1, wherein said acylation group is selected from the group consisting of N-hexadecandioyl-gamma-L-glutamyl, N-octadecandioyl-gamma-L-glutamyl-[2-(2-amino-ethoxy)-ethoxy]-acetyl-[2-(2-amino-ethoxy)-ethoxy]-acetyl, N-eicosandioyl-gamma-L-glutamyl-[2-(2-amino-ethoxy)-ethoxy]-acetyl-[2-(2-amino-ethoxy)-ethoxy]-acetyl, and N-docosandioyl-gamma-L-glutamyl-[2-(2-amino-ethoxy)-ethoxy]-acetyl-[2-(2-amino-ethoxy)-ethoxy]-acetyl.

7. The IL-1Ra compound according to claim 1, wherein said acylation group is attached to IL-1Ra via the epsilon amino group of a Lys amino acid residue of said IL-1Ra or the an amino group of a N-terminal amino residue.

8. The IL-1Ra compound according to claim 1, wherein said IL-1Ra comprises a substitution selected from the group consisting of K31R, K34R, K118R and K121R relative to hIL-1Ra Isoform 1.

9. The IL-1Ra compound according to claim 1, wherein said IL-1Ra compound is selected from the group consisting of N-epsilon118-[N-hexadecandioyl-gamma-L-glutamate][Met25]hIL-1Ra(25-177) (compound A1), N-epsilon1214N-hexadecandioyl-gamma-L-glutamate][Met25]hIL-1Ra(25-177) (compound A2), N-epsilon118-[N-octadecandioyl-gamma-L-glutamyl-[2-(2-amino-ethoxy)-ethoxy]-acetyl-[2-(2-amino-ethoxy)-ethoxy]-acetyl][Met25]hIL-1Ra(25-177) (compound B), N-epsilon31-[N-octadecandioyl-gamma-L-glutamyl-[2-(2-amino-ethoxy)-ethoxy]-acetyl-[2-(2-amino-ethoxy)-ethoxy]-acetyl][Met25]hIL-1Ra(25-177) (compound C1), N-epsilon34-[N-octadecandioyl-gamma-L-glutamyl-[2-(2-amino-ethoxy)-ethoxy]-acetyl-[2-(2-amino-ethoxy)-ethoxy]-acetyl][Met25]hIL-1Ra(25-177) (compound C2), N-epsilon118-[N-eicosandioyl-gamma-L-glutamyl-[2-(2-amino-ethoxy)-ethoxy]-acetyl-[2-(2-amino-ethoxy)-ethoxy]-acetyl][Met25]hIL-1Ra(25-177) (compound D1), N-epsilon121-[N-eicosandioyl-gamma-L-glutamyl-[2-(2-amino-ethoxy)-ethoxy]-acetyl-[2-(2-amino-ethoxy)-ethoxy]-acetyl][Met25]hIL-1Ra(25-177) (compound D2), a mixture of compound A1 and A2, a mixture of compound C1 and C2, a mixture of compound B, C1, C2, and a mixture of compound D1 and D2.

10. A composition comprising the IL-1Ra compound as defined in claim 1 and one or more excipients.

11. (canceled)

12. A nucleotide sequence comprising a sequence selected from the group consisting of construct no. 2, construct no. 3, construct no. 4, construct no. 5, construct no. 6, construct no. 7 and construct no. 8.

13. A vector comprising the nucleotide sequence as defined in claim 12.

14. A host cell comprising the nucleotide sequence as defined in claim 12.

15. A method for the preparation of an IL-1Ra compound, said method comprising the step of expressing recombinantly the nucleotide sequence as defined in claim 12, at a temperature below 25° C.

16. The method of claim 15, wherein the temperature below 25° C. is 18° C.
